(19) United States
(12) Patent Application Publication
(10) Pub. No.: US 2023/0145555 A1
(43) Pub. Date: May 11, 2023

(54) COMPOSITION COMPRISING RESVERATROL

(71) Applicant: GALENUS G.H. AG, Hergiswil (CH)

(72) Inventors: Rita DOBMEYER, Hergiswil (CH); Kurt NABER, Straubing (DE)

(21) Appl. No.: 17/917,608

(22) PCT Filed: Mar. 19, 2021
(86) PCT No.: PCT/EP2021/057161

§ 371 Date: Oct. 7, 2022

(30) Foreign Application Priority Data
Apr. 10, 2020 (EP) 20169221.7

(51) Int. Cl. A61K 31/05; A61K 31/593; A61P 31/14; A61K 9/00; A61K 47/12; A61K 47/10

(52) U.S. Cl. CPC A61K 31/05; A61K 31/593; A61P 31/14; A61K 9/0043; A61K 9/006; A61K 47/12; A61K 47/10

(57) ABSTRACT

The invention relates to a composition comprising an effective amount of a source of a resveratrol derivative and an effective amount of a vitamin D for the prevention of an acute respiratory distress syndrome and/or an acute lung injury caused by SRS-CoV-2 and to a kit for the preparation of said composition.

### CROSS-REFERENCE TO RELATED APPLICATIONS

This application claims the benefit of priority to European patent application EP20169221.7 filed Apr. 10, 2020, which is incorporated by reference herein in its entirety.

### BACKGROUND OF THE INVENTION

### 1. Technical Field

The invention relates to a composition consisting essentially of an effective amount of a source of a resveratrol derivative, an effective amount of a vitamin D, and an acceptable excipient for the prevention of an acute respiratory distress syndrome and/or an acute lung injury caused by SARS-CoV-2 and to a kit for the preparation of said composition.

### 2. Prior Art

Critically ill COVID-19 patients often develop acute respiratory distress syndrome and acute lung injury (ARDS/ALI). The uncontrolled progressive inflammation in the lungs causes acute diffuse alveolar damage recognized as areas with ground-glass opacities, and other areas with increased density but without any recognizable vessels (consolidation), J. Gattinoni et al. Am. J. Respir. Crit. Care Med., Vol. 164, pp. 1701-1711, 2001DOI: 10.1164/rccm2103121.

When ARDS progresses to the acute phase, alveolar flooding (edema), interstitial inflammation and compression atelectasis, as well as increase in lung tissue and reduction in lung gas volume are observed, L. PuybassetP. et al. Intensive Care Med 26, 857-869 (2000)—DOI: 10.1007/s001340051274

COVID-19 patients suffering from ARDS/ALI often require intubation and invasive mechanical ventilation to assist difficulty in breathing because the increasing hypoxemic respiratory failure results in acute diffuse alveolar damage, S. A. Ňamendys-Silva, Lancet Respir. Med. 2020, Mar. 5, 2020—DOI: Published:Mar. 05, 2020DOI: 10.1016/S2213-2600(20)30110-7.

Acute respiratory distress syndrome and acute lung injury (ARDS/ALI) are often characterized by the accumulation of neutrophils in the lungs and the increased production of inflammatory cytokines, chemokines, proteases and oxidants. The initiation and development of ARDS/ALI is dependent upon the activation of inflammasomes, which are an integral part of our innate immune system.

Inflammasomes sense pathogens, danger associated molecular patterns (DAMPs) as well as biological crystals including urate and cholesterol. The activation of inflammasomes releases pro-inflammatory cytokines interleukin (IL)-1β and IL-18.

Recently, the NLRP3 inflammasome has been identified as key to the induction of ADRS/ALI Regulation of the NLRP3 inflammasome and macrophage pyroptosis by the p38 MAPK signaling pathway in a mouse model of acute lung injury, Dandan Li et al. Mol. Med. Rep. 2018 Nov; 18(5): 4399-4409—DOI: 10.3892/mmr.2018.9427].

Interleukin 1 beta (IL-1β) is a potent proinflammatory cytokine that is implicated in the pathogenesis of acute respiratory distress syndrome because the initiation of hypoxemia (below normal oxygen levels in blood) is induced by IL-1β signaling. The production of IL-1β is tightly controlled and is dependent upon NLRP3 inflammasome activation.

The NLRP3 inflammasome is a caspase-1-containing multi-protein complex that controls the release of IL-1β and plays important roles in the development of inflammatory disease. It has been shown that resveratrol, a polyphenolic compound naturally produced by plants, inhibits NLRP3 inflammasome-derived IL-1β secretion and pyroptosis in macrophages. Resveratrol inhibits the activation step of the NLRP3 inflammasome by suppressing mitochondrial damage. Resveratrol also induces autophagy by activating p38, and macrophages treated with an autophagy inhibitor are resistant to the suppressive effects of resveratrol, Y. P. Chang et al. J Cell Physiol. 2015 Jul; 230(7):1567-79—DOI: 10.1002/jcp.24903.

Vitamin D deficiency has a relationship with the incidence of asthma, as shown by epidemiological information. Studies indicate that vitamin D has anti-inflammatory effects on chronic lung inflammation and is a potential regulator of the development of respiratory diseases including asthma and chronic obstructive pulmonary disease, J. H. Zhang et al. Braz. J. Med. Biol. Res. 2018; 51(1): e6841—DOI: 10.1590/1414-431X20176841.

Moreover, NLRP3 inflammasome is also critical in the development of allergic airway inflammation.

In view of the global pandemic spreading of SARS-CoV-2 there is an essential need and high demand for a medication, which prevents the outbreak of ARS/ALI in infected persons and/or protects uninfected persons from infection.

Up to now there is no such medication. Accordingly, the problem underlying the present invention is to provide a medication for the prevention of an acute respiratory distress syndrome and/or an acute lung injury caused by SARS-CoV-2.

### SHORT SUMMARY OF THE INVENTION

Surprisingly, it has been found, that a composition comprising an effective amount of a source of a resveratrol derivative and an effective amount of a vitamin D efficiently prevents the acute respiratory distress syndrome and/or an acute lung injury in patients infected by SARS-CoV-2, in particular in infected, but still asymptomatic or only mildly symptomatic patients.

Accordingly, the present invention relates to a composition consisting essentially of a pharmaceutically effective amount of a source of a resveratrol derivative, a pharmaceutically effective amount of a vitamin D, and a pharmaceutically acceptable excipient for the prevention of an acute respiratory distress syndrome and/or an acute lung injury caused by SARS-CoV-2.

Furthermore, the invention relates to a kit of parts for the preparation of a composition in accordance with the invention essentially consisting of

(i) a first compartment containing a composition, which is in a form suitable for oral, local or inhalative administration, comprising an effective amount of a source of a resveratrol derivative, and an acceptable excipient;

(ii) a second compartment containing a composition, which is in a form suitable for oral, local, inhalative or parenteral administration, comprising an effective amount of a vitamin D, and a pharmaceutically acceptable excipient;

(iii) optionally a leaflet describing the dosage and administration of each of the compositions (i) and (ii).

Furthermore, the invention relates to a method for the prevention of an acute respiratory distress syndrome and/or an acute lung injury caused by SARS-CoV-2 in a person in need of such prevention, which method comprises administering simultaneously or subsequently effective amounts of
(a) an effective amount of a source of a resveratrol derivative, and
(b) an effective amount of a vitamin D.

Another aspect of the invention is resveratrol for the prevention of an asymptomatic or mild viral infection of the respiratory system by the coronavirus 2 (SARS-CoV-2), wherein an effective amount of resveratrol is administered locally to the to the respiratory system, preferably by inhalation, in particular by a nasal or throat spray.

### SHORT DESCRIPPTION OF THE DRAWING

FIG. 1 is a diagram in which the results of the tests in the K18-hACE2 transgenic mouse model (cp. Example 1) is shown.

### DETAILED DESCRIPTION OF THE INVENTION

The term “source of a resveratrol derivative” as used hereinbefore and herein below embraces the sources of natural occurring hydroxylated stilbenes or stilbenoids including the methyl ethers and glycosides thereof. Preferred resveratrol derivatives are resveratrol, oxy-resveratrol also known as piceatannol, the methylated stilbenoids selected from the group consisting of 4-methoxyresveratrol, gnetucleistol D (2-methoxyoxyresveratrol), gnetucleistol E (3-methoxy-isorhapontigenin), isorhapontigenin (3,4′,5-trihydroxy-3′-methoxystilbene), pinostilbene (3-methoxyresveratrol), pterostilbene (3′,5′-dimethoxyresveratrol), rhapontigenin (piceatannol 4′-methyl ether), combretastatin A-1 and combretastatin A-4, and the glycosylated stilbenoids selected from the group consisting of piceid (trans-resveratrol-3-O-glucoside), trans-resveratrol-3-O-glucuronide, resveratroloside (trans-resveratrol-4′-O-beta-D-glucopyranoside), rhapontigenin 3-O-rutinoside, 4′-methoxy-(E)-resveratrol 3-O-rutinoside and rhaponticin (rhapontigenin glucoside).

Most preferred are resveratrol of formula (IA) and oxyresveratrol of formula (IB):

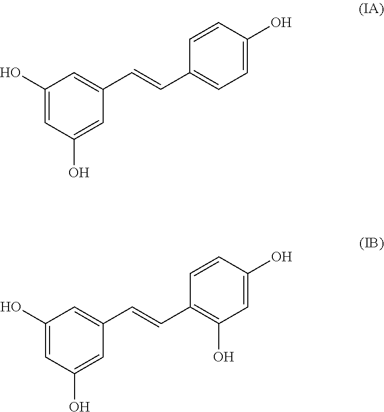 [SMILES: Oc1ccc(/C=C/c2cc(O)cc(O)c2)cc1 ; Oc1cc(O)cc(/C=C/c2ccc(O)cc2O)c1]

It is well known that resveratrol (IA) is almost insoluble in water and has stability issues. T. Petek et al., Molecules 2017, 22, 137; doi 10.3390 suggest increasing the solubility and stability of resveratrol in water with the aid of a dendrimer nanotechnology or liposomal resveratrol preparations. In addition, the solubility of resveratrol in water may be enhanced by associating it with carboxymethyl beta-glucan as suggested by the European Patent EP 2 674 155 B1.

The term “vitamin D” as used hereinbefore and herein below relates to a group of fat-soluble secosteroids responsible for increasing intestinal absorption of calcium, magnesium, and phosphate, and multiple other biological effects. The most important compounds in this group are vitamin D₃ (also known as cholecalciferol), vitamin D₂ (ergocalciferol) and calcitriol (1,25-dihydroxycholecalciferol), which is the hormonally active metabolite of vitamin D. Only a few foods contain vitamin D. The major natural source of the vitamin is synthesis of cholecalciferol in the skin from cholesterol through a chemical reaction that is dependent on sun exposure (specifically UVB radiation).

Most preferred are vitamin D3 of formula (IIA) and calcitriol of formula (IIB)

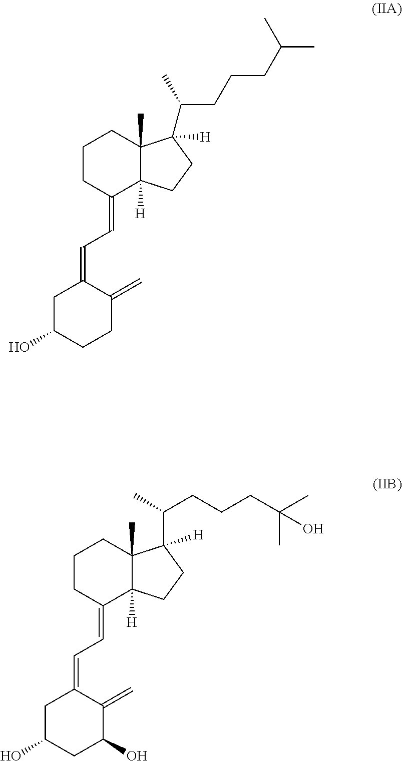 [SMILES: C=C1CC[C@H](O)C/C1=C/C=C1\CCC[C@]2(C)[C@@H]([C@H](C)CCCC(C)C)CC[C@@H]12 ; C=C1/C(=C\C=C2/CCC[C@]3(C)[C@@H]([C@H](C)CCCC(C)(C)O)CC[C@@H]23)C[C@@H](O)C[C@@H]1O]

The term “pharmaceutically acceptable salts” is meant to include salts of the compounds of the invention which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66: 1-19 (1977)). Certain specific compounds of the present invention contain both basic and acidic functionalities that allow the compounds to be converted into either base or acid addition salts.

As used herein, the term “acceptable” relates to molecular entities and compositions that are physiologically tolerable and do not normally cause an allergic reaction or a similar adverse reaction, such as gastric discomfort, dizziness and the like, when administered to humans. As used herein, the term “acceptable” preferably means pharmaceutically acceptable, acceptable for medicinal products or devices or, nutraceutically acceptable. Furthermore, pharmaceutically acceptable, means that it is approved by a regulatory agency of the federal or state government or listed in the US pharmacopoeia or another pharmacopoeia, generally recognized for its use in animals, preferably in mammals and more particularly in human beings.

Pharmaceutical compositions are and preparations made from substances that are used to heal or alleviate illnesses or to ensure that illnesses or complaints do not occur in the first place. This applies to both human and animal use. The substances can act in the body or on the body.

Compositions for medical devices (medical motto) are products with a medical purpose that are intended by the manufacturer for human use. In contrast to pharmaceutical compositions that have a pharmacological, immunological or metabolic effect, the intended main effect in medical devices is primarily achieved for example physically.

Neutraceutical compositions or dietary supplements are foods. Therefore, on the one hand, they are subject to the extensive legal provisions that apply to all foods. On the other hand, there are additional, special regulations for food supplements, e.g., regarding to composition and labeling. At the European level, these requirements are primarily contained in Directive 2002/46/EC on food supplements.
They are intended to supplement the general diet,
They consist of natural products, e.g., vitamins or minerals or other substances with a nutritional or physiological effect and are in concentrated form;
They are put on the market in dosed form for absorption in measured small quantities.

As used herein, the term “in combination with” or “co-administered” covers both separate and sequential administration of the active agents. For example, when the agents are administered sequentially, either resveratrol or vitamin D may be administered first. When administration is simultaneous, the agents may be administered either in the same or a different pharmaceutical composition. Adjunctive therapy, i.e. where one agent is used as a primary treatment and the other agent is used to assist that primary treatment, is also an embodiment of the present invention.

By “effective” amount of an active principle, a drug, formulation, or permeant is meant a sufficient amount of the active principle to provide the desired local or systemic effect. A “topically effective,” “cosmetically effective,” “pharmaceutically effective”, dietary effective“, medicinal effective”, “preventively effective”,or “therapeutically effective” amount refers to the amount of an active ingredient needed to effect the desired effect.

The term “synergistically effective” amount of a combination of active ingredients means that the effect of the combination of the active principles is higher than the sum of the expected effects of both the active ingredients, when being administered alone. In a preferred embodiment the source of a resveratrol derivative and a vitamin D are administered in synergistically effective amounts.

The term “coronavirus SARS-CoV-2” as used hereinbefore or hereinbelow means virus that causes coronavirus disease 2019 (COVID-19), the respiratory illness responsible for the COVID-19 pandemic. Colloquially known as simply the coronavirus, it was previously referred to by its provisional name, 2019 novel coronavirus (2019-nCoV), and has also been called human coronavirus 2019 (HCoV-19 or hCoV-19). It includes several variants or mutants, in particular those notable variants of SARS-CoV-2, which emerged in the fall of 2020.
The Variant of Concern 202012/01 (VOC 202012/01) or B.1.1.7 is believed to have emerged in the United Kingdom in September. Among the variant's several mutations is one in the receptor-binding domain of the spike protein that changes the asparagine at position 501 to tyrosine (N501Y).
The 501Y.V2 Variant, which has the same N501Y mutation, arose independently in South Africa. It was detected in patient specimens collected at the beginning of October 2020.
The B. I.207 variant appeared in Nigeria. It has a mutation in the spike protein (P681H) that is also found in the VOC 202012/01 variant. P681H is located near the S1/S2 furin cleavage site.
The Cluster 5 variant emerged among minks and mink farmers in Denmark. It has a set of mutations that have not been observed in other variants, including four amino acid changes in the spike protein.
Th Brazilian variant (P.1), which was detected in Manaus on Jan 12, 2021 with ten unique spike protein mutations, including E484K and N501K.
Moreover, the newly classified P.2 lineage (sub-lineage of B.1.128 that independently accrued the spike E484K mutation) with the E484K mutation have been detected in two people who have been reinfected with SARS-CoV-2 in Brazil.

The term “asymptomatic or mild viral infection” as used hereinbefore or hereinbelow means that the coronavirus SARS-CoV-2, which is spread by direct contact or aerosols directly from one person to another person or by indirect contact with objects, which have been contaminated by an infected person, has infected the mucous membranes of the respiratory system, but the symptoms of the disease caused by said virus have not yet emerged or are only mild. The typical symptoms of COVID-19 such as fever or chills, cough, shortness of breath or difficulty breathing, fatigue, muscle or body aches, headache, loss of taste or smell, sore throat, congestion or runny nose, nausea or vomiting, diarrhea will not have evolved even though the patient has been tested positive. Under these circumstances, the virus will be located on the mucous membrane of the respiratory system, the surface of the epithelial cells or within the epithelial cells without having entered further cells of the host.

The term “respiratory system” as used hereinbefore or hereinbelow means the biological system consisting of specific organs and structures used for gas exchange in mammals, in particular human beings. The respiratory tract is divided into an upper and a lower respiratory tract. The upper tract includes the nose, nasal cavities, sinuses, oral cavity, pharynx and the part of the larynx above the vocal folds. The lower tract includes the lower part of the larynx, the trachea, bronchi bronchioles and the alveoli in the lungs.

The composition according to the invention includes pharmaceutical compositions, compositions for medical devices and nutraceutical compositions.

Further embodiments of the invention are the following compositions:

(A) wherein the source of a resveratrol derivative is selected from the group consisting of synthetically produced hydroxylated stilbenes, liposomal resveratrol and extracts of plants comprising high amounts of resveratrol;

(B) wherein the source of a resveratrol derivative is selected from the group consisting the resveratrol of formula (IA) and hydroxyresveratrol of formula (IB), and an extract of polygonum cuspidatum or polygonum aviculare.

(C) wherein the vitamin D is selected from the group consisting of vitamin D₃ and calcitriol;

(D) which is suitable for the prevention of an acute respiratory distress syndrome and/or an acute lung injury caused by SARS-CoV-2 by inhibiting the NLRP3 inflammasome;

(E) which comprises 0.001 to 10% by weight, preferably 0.02 to 5%, in particular 0.05 to 1% by weight of a source of a resveratrol derivative, in particular resveratrol of formula IA and/or 0.025 mg (1′000 IE) to 0.50 mg (20′000 IE) of a vitamin D, in particular 0.05 (2′000 IE) to 0.1 (4′000 IE) mg of vitamin D, in particular vitamin D₃ or calcitriol, each with respect to the total weight of the composition.

(F) which is in a form suitable for oral administration.

(G) which is in a form suitable for local administration by nasal drops, nasal spray, throat spray, mouth wash or inhalation.

The term “inhalation” as used herein before and herein below means the administration of a source of a resveratrol derivative and or a vitamin D in form of an aerosol by a process, which begins with the contraction of the muscles attached to the rib cage; this causes an expansion in the chest cavity. Then takes place the onset of contraction of the diaphragm, which results in expansion of the intrapleural space and an increase in negative pressure according to Boyle's law. This negative pressure generates airflow because of the pressure difference between the atmosphere and alveolus. The inhalation may be carried out with the aid of a nasal spray, a mouth spray, a throat spray or an inhalation device.

Preferably such composition for inhalational administration contains an acceptable excipient comprising a solvent system consisting essentially of one or more components selected from the group consisting of water, one or more alcohols, one or more hydrophobic solvents, one or more surfactants, one or more emulsifiers, one or more agents for the adjustment of the pH, one or more thickeners and one or more nanoparticle forming polymers.

(H) which is in a form suitable for inhalational administration by an inhaler selected from the group consisting of nasal sprays, oral sprays, throat sprays, propellant-free soft mist inhalers, in particular the Respimat®, pressurized metered dose inhalers (p-MDI) and dry powder inhalers (DPI) (cp. H. Wachtel et al. Pulm. Ther. (2017) 3:19-230).

Another embodiment of the invention is the pharmaceutical kit according to the invention

(I) comprising two separate compartments:

(i) a first compartment containing a composition, which is in a form suitable for oral, local or inhalative administration, comprising 0.001 to 10% by weight, preferably 0.05 to 5% by weight of a source of a resveratrol derivative, in particular resveratrol of formula IA, and an acceptable excipient;

(ii) a second compartment containing a composition, which is in a form suitable for oral, local, inhalative or parenteral administration, comprising 0.025 (1′000 IE) to 0.50 (20′000 IE) mg of a vitamin D, in particular vitamin D₃, and an acceptable excipient;

Dosage levels of the combined active ingredients are in the order of from about 1.0 mg to about 250 mg per kilogram of body weight per day and more preferably from about 2.5 mg to about 150 mg per kilogram of body weight per day, are useful in the treatment of the above-indicated conditions. The amount of each active ingredient that may be combined with the carrier materials to produce a single dosage form will vary depending upon the condition being treated and the mode of administration. Dosage unit forms will generally contain between from about 1 mg to about 500 mg of each of the active ingredients.

Frequency of dosage may also vary depending on the compound used and the severity of the infection to be treated. However, for treatment of most infections, a dosage regimen of 4 times daily or less is preferred. It will be understood, however, that the specific dose level for any patient will depend upon a variety of factors including the activity of the specific compound employed, the age, body weight, general health, sex, diet, time of administration, route of administration and rate of excretion, drug combination and the severity of the particular infection undergoing therapy.

The compositions of the invention may be administered orally, topically, parenterally, by inhalation or spray or rectally in dosage unit formulations containing conventional non-toxic pharmaceutically acceptable carriers, adjuvants and vehicles. It is further understood that the best method of administration may be a combination of methods. Oral administration in the form of a pill, capsule, elixir, syrup, lozenge, troche, or the like is particularly preferred. Further preferred is the local administration to the upper respiratory tract, preferably by inhalation, in particular by nasal, mouth or throat sprays, or by administration of nasal, mouth or throat drops. The term parenteral as used herein includes sub-cutaneous injections.

The formulations may conveniently be presented in unit dosage form and may be prepared by any of the methods well known in the art of pharmacy e.g. as described in “Remington: The Science and Practice of Pharmacy”, Lippincott Williams and Wilkins, 21^{st} Edition, (2005). Suitable methods include the step of bringing into association to active ingredients with a carrier which constitutes one or more excipients. In general, formulations are prepared by uniformly and intimately bringing into association the active ingredients with liquid carriers or finely divided solid carriers or both and then, if necessary, shaping the product into the desired formulation. It will be appreciated that when the two active ingredients are administered independently, each may be administered by a different means.

Formulations suitable for oral administration may be presented as discrete units such as capsules, cachets or tablets (e.g. chewable tablets in particular for pediatric administration), each containing a predetermined amount of active ingredient; as powder or granules; as a solution or suspension in an aqueous liquid or non-aqueous liquid; or as an oil-in-water liquid emulsion or water-in-oil liquid emulsion. The active ingredients may also be presented a bolus, electuary or paste.

Alternatively, the active ingredients may be incorporated into oral liquid preparations such as aqueous or oily suspensions, solutions, emulsions, syrups, or elixirs. Formulations containing the active ingredients may also be presented as a dry product for constitution with water or another suitable vehicle before use. Such liquid preparations may contain conventional additives such as suspending agents (e.g. sorbitol syrup, methyl cellulose, glucose/sugar syrup, gelatin, hydroxymethyl cellulose, carboxymethyl cellulose, aluminum stearate gel and/or hydrogenated edible fats), emulsifying agents (e.g. lecithin, sorbitan mono-oleate and/or acacia), non-aqueous vehicles (e.g. edible oils, such as almond oil, fractionated coconut oil, oily esters, propylene glycol and/or ethyl alcohol), and preservatives (e.g. methyl or propyl p-hydroxybenzoates and/or sorbic acid).

Chlorofluorocarbons (CFC) or hydrofluoroalkanes (HFA) can be used to generate the energy to produce inhalable drug particles from liquid formulations in p-MDIs. DPIs are breath actuated and use the energy of inspiration to disperse the dry powder and transport the inhaled medication to the lungs.

In a soft mist inhaler the energy to generate a fine mist stems from a mechanical spring, which forces the metered drug solution through channels to produce two jets of liquid converging at a controlled angle as described in U.S. Pat. No. 5,497, 944 and in the International Patent Applications WO 91/14468 and WO 97/12687 (cf. in particular FIGS. 6a and 6b).

In general, formulations for inhalation are prepared by uniformly and intimately bringing into association the active ingredients with liquid carriers or finely divided solid carriers or both and then, filling the product into the desired inhaler. It will be appreciated that when the two active ingredients are administered independently, each may be administered by a different means.

Various known devices may be used to administer pulmonary formulations, whether dry powder, aerosol or liquid. Dry powder inhalers (DPI) are well known to those skilled in the art and are used to administer the aforementioned dry powder formulations. Suitable dry powder inhalation devices for administering the present formulations include, for example, TURBOHALER® (Astra Pharmaceutical Products, Inc., Westborough, Mass.), ROTAHALER® (Allen & Hanburys, Ltd., London, England). Aerosol formulations may be administered via pressurized metered-dose inhalers (p-MDI). Liquid formulations of the invention may be administered via a pump spray bottle, propellant-free soft mist inhalers, or nebulizer. Suitable liquid inhalation devices for administrating the present formulations include, for example, Compressor Nebulizer C102 Total (NE-C102-E), (3A HEALTH CARE S.r.I., Via Marziale Cerutti, 90F/G, 25017 Lonato del Garda (BS), Italy).

The DPI formulation according to the invention consists of micronized, spray-dried, or nanonized resveratrol and/or vitamin D blended with larger carrier particles, which enhance flow, reduce aggregation, and aid in dispersion. Suitable methods and carrier particles are described by M. J. Telko and A. J. Hickey, Respiratory Care September 2005, 50 (9) 1209-1227.

As a rule, the carrier is a selected from the group consisting of carbohydrates, reduced carbohydrates, and phospholipids. Preferred carriers for DPI are lactose, mannitol, glucose, and mixtures thereof.

The formulation of resveratrol and/or vitamin D for p-MDI may also contain co-solvents, preferably water or one or more alcohols including polyalcohols, more preferably one or more non-branched or branched C₂₋₅ alcohol, in particular ethanol, glycerin or propylene glycol. In addition, the formulation for p-MDI may contain one or more excipients selected from the group consisting of antioxidants, preservatives, emulsifiers, dispersants, surfactants, chelating agents, pH adjusting agents, buffering agents, preferably citric acid.

Formulations for p-MDI comprising resveratrol and/or vitamin D may be prepared using propellants, co-solvents and further ingredients in analogy to any of the methods described by P. B. Myrdal et al., AAPS PharmSciTech. 2014 Apr; 15(2): 434-455, doi: 10.1208/s12249-013-0063-x.

The following propellants may be used in the p-MDI formulation according to the invention: trichlorofluoromethane (CFC 11), dichlorodifluoromethane (CFC 12), dichlorotetrafluoroethane (CFC 114), 1,1,1,2-tetrafluoroethane (HFA 134a) and 1,1,1,2,3,3,3-heptafluoropropane (HFA 227), 1,1-difluoroethane (HFA 152a), propane, n-butane, isobutane, n-pentane, isopentane, neopentane, dimethylether, and hydrofluoro-olefins (HFO) or blends of these propellants. Most preferred are HFA 134a and HFA 227.

The formulation of resveratrol and/or vitamin D propellant free soft mist inhaler such as the Respimat® comprise a solvent system in which one or both active ingredients are dissolved or suspended. As a rule, the solvent system is an admixture of water, co-solvents and/or other excipients and additives that may be added to the propellant-free inhalable solutions which may be used according to the invention. Preferred co-solvents are those which contain hydroxyl groups or other polar groups, e.g., alcohols, particularly ethanol, isopropyl alcohol, glycols, particularly propyleneglycol, polyethyleneglycol, polypropyleneglycol, glycolether, glycerol, polyoxyethylene alcohols and polyoxyethylene fatty acid esters. The excipients and additives include, for example, surfactants such as soya lecithin, oleic acid, sorbitan esters, such as polysorbates, polyvinylpyrrolidone, other stabilizers, complexing agents, antioxidants and/or preservatives which guarantee or prolong the shelf life of the finished pharmaceutical formulation, flavorings, vitamins and/or other additives known in the art. The additives also include pharmacologically acceptable salts such as sodium chloride as isotonic agents.

An apparatus of this kind for the propellant-free administration of a metered amount of a liquid pharmaceutical composition for inhalation is described in detail for example in International Patent Applications WO 91/14468 and WO 97/12687, cf. FIGS. 6a and 6b and the accompanying description. In a nebulizer of this kind a pharmaceutical solution is converted by means of a high pressure of up to 600 bar into an aerosol destined for the lungs, which is sprayed. Within the scope of the present specification, reference is expressly made to the entire contents of the literature mentioned above.

The preferred atomizer essentially consists of an upper housing part, a pump housing, a nozzle, a locking clamp, a spring housing, a spring, and a storage container, characterized by
a pump housing fixed in the upper housing part and carrying at one end a nozzle body with the nozzle or nozzle arrangement,
a hollow piston with valve body,
a power take-off flange in which the hollow body is fixed, and which is located in the upper housing part,
a locking clamping mechanism located in the upper housing part,
a spring housing with the spring located therein, which is rotatably mounted on the upper housing part by means of a rotary bearing
a lower housing part which is fitted onto the spring housing in the axial direction.

At the moment of release of the spring the hollow piston with valve body exerts, at its high-pressure end, a pressure of 5 to 60 MPa (about 50 to 600 bar), preferably 10 to 60 MPa (about 100 to 600 bar) on the fluid, the measured amount of active substance solution at the moment when the spring is actuated. Volumes of 10 to 50 microliters are preferred, volumes of 10 to 20 microliters are more preferred, whilst a volume of 15 microliters per actuation is particularly preferred.

In inhalers of this kind the formulations of solutions are stored in a reservoir. It is essential that the active substance formulations used are sufficiently stable when stored and at the same time are such that they can be administered directly, if possible, without any further handling, in accordance with their medical purpose. Moreover, they must not contain any ingredients which might interact with the inhaler in such a way as to damage the inhaler or the pharmaceutical quality of the solution or of the aerosol produced.

The formulation for the nasal and throat spray according to the invention may be prepared in analogy of the methods described in the Chinese patent application CN 103830355 A or the European patent EP 2 674 155 B1 and comprises resveratrol and/or vitamin D, water, Tween®80, sodium carboxymethyl cellulose, carboxymethyl glucan and/or a buffer.

A nasal spray containing resveratrol, which can be used as a first component of a pharmaceutical kit is commercially available from Noos S.r.1., Roma, Italy under the tradename LinfoVir® plus nasal spray.

An oral spray containing 800 IU vitamin D₃ in two spray doses, which can be used as a second component of a pharmaceutical kit, is commercially available in form of a 10 ml spray is commercially available from Feelgood Shop B. V., Venlo, the Netherlands under the name LIPOSOMAL VITAMIN D3 Spray

Compositions for use according to the invention may be presented in a pack or dispenser device which may contain one or more unit dosage forms containing the active ingredients. The pack may, e.g., comprise a glass vial, a metal or plastic foil, such as a blister pack. Where the compositions are intended for administration as two separate compositions these may be presented in the form of a twin pack or a kit.

The compositions according to the invention may also be prescribed to the patient in kit or “patient packs” containing the whole course of treatment in a single package, usually a blister pack or a pack of glass vials. Patient packs have an advantage over traditional prescriptions, where a pharmacist divides a patients' supply of a pharmaceutical from a bulk supply, in that the patient or the treating health professional always has access to the package insert contained in the patient pack, normally missing in traditional prescriptions. The inclusion of the package insert has been shown to improve patient compliance with the physician's instructions. The administration of the combination of the invention by means of a single patient pack, or patient packs of each composition, including a package insert directing the patient to the correct use of the invention is a desirable feature of this invention.

According to a further embodiment of the present invention there is provided a kit comprising at least one active ingredient of the combination according to the invention and an information insert containing directions on the use of the combination of the invention.

Dosages of vitamin D₃ as solution for parenteral administration are commercially available for example as Vitamin D₃ Streuli® from Streuli Pharma AG, 8730 Uznach, Switzerland. This product contains in an ampoule (1 mL) 4,000 IU of cholecalciferol.

Moreover, the quantity of the compound or composition of the present invention administered will vary depending on the patient and the mode of administration and can be any effective amount.

In an exemplary embodiment, 0.001 to 10% by weight of a resveratrol derivative are present in a pharmaceutical unit dose according to the invention. In an exemplary embodiment, 0.05 to 5% by weight of a resveratrol derivative are present in a pharmaceutical unit dose according to the invention. In an exemplary embodiment, 0.1 to 4% by weight of a resveratrol derivative are present in a pharmaceutical unit dose according to the invention. In an exemplary embodiment, 0.15 to 3.5% by weight of a resveratrol derivative are present in a pharmaceutical unit dose according to the invention. In an exemplary embodiment, 0.2 to 2.5% by weight of a resveratrol derivative are present in a pharmaceutical unit dose according to the invention.

Doses of resveratrol, in particular of formula IA, employed for systemic treatment of adult humans will typically be in the range of 100 mg to 1000 mg, preferably 150 mg to 900 mg, in particular 200 mg to 850 mg per day.

Doses of resveratrol, in particular of formula IA, employed for local treatment of the respiratory system of adult humans will typically be in the range of 1.0 mg to 100 mg, preferably 1.5 mg to 50 mg, in particular 2.0 mg to 20 mg per day.

Doses of vitamin D, in particular vitamin D₃, employed for adult human treatment will typically be in the range 0.025 mg (1′000 IE) to 0.50 mg (20′000 IE) per day, preferably 0.0375 mg (1′500 IE) to 0.25 mg (10′000 IE), in particular 0.05 mg (2′000 IE) to 0.10 mg (4′000 IE) per day.

In a first exemplary embodiment, the patient is a human, who (i) bears the risk to become infected by SARS-CoV-2, is infected by SARS-CoV-2 (ii) without or (iii) with respiratory symptoms.

In the different groups different doses of resveratrol and vitamin D will be administered:

Systemic Resveratrol:
(i) Uninfected: 100 mg to 600 mg per day;
(ii) Infected w/o respiratory symptoms 200 mg to 800 mg, per day;
(iii) Infected with mild respiratory symptoms 400 mg to 1000 mg per day.

Resveratrol locally administered to the respiratory system:
(i) Uninfected: 1.0 mg to 10.0 mg per day;
(ii) Infected w/o respiratory symptoms 2.0 mg to 20.0 mg, per day;
(iii) Infected with mild respiratory symptoms 6.0 mg to 50.0 mg per day.

Vitamin D:
(i) Uninfected: 0.025 mg (800 IE) to 0.10 mg (4′000 IE) per day;
(ii) Infected w/o respiratory symptoms: 0.0375 mg (1′500 IE) to 0.25 mg (10′000 IE) per day;
(iii) Infected with respiratory symptoms 0.05 mg (2′000 IE) to 0.10 mg (20′000 IE) per day.

In another exemplary embodiment, the patient is a member selected from a human and any mammal, which may become infected by SARS COV-2 including cattle, goat, pig, sheep, horse, cow, bull, dog, guinea pig, gerbil, rabbit, and cat. Most preferably the patient is a human of any gender or age. Since all the components of the pharmaceutical composition of the invention are compatible for elderly people, it can be used for the treatment of geriatric patients. The term “geriatric patients” as used hereinbefore and hereinbelow includes senior people of an age of 60 years onwards, who may be or may become infected by the coronavirus 2 and may suffer from severe symptoms of SARS CoV-2 without preventive treatment.

### EXEMPLIFICATION

The invention now being generally described, will be more readily understood by reference to the following Examples, which are included merely for purposes of illustration of certain aspects and embodiments of the present invention, and are not intended to limit the invention.

All publications and patents mentioned herein are hereby incorporated by reference in their entirety as if each individual publication or patent was specifically and individually indicated to be incorporated by reference. In case of conflict, the present application, including any definitions herein, will control.

### Example 1: Mouse Model

Introducing the K18-hACE2 Transgenic Mouse for Coronavirus Research

A vector carrying a human ACE2-coding sequence is introduced into wild-type mice subsequently developing a hACE2 transgenic mouse strain, as described by P. B. McCray et al. J Virol. 2007 Jan; 81(2): 813-821—DOI: 10.1128/JVI.02012-06. ACE2 expression, which is regulated by the human cytokeratin 18 (K18) promoter in epithelial cells, is observed in the initially infected airway epithelial cells.

The infection will begin in the airway epithelium, spread to the alveoli and finally out of the lungs to the brain. The infection causes infiltration of macrophages and lymphocytes in the lungs and up-regulation of pro-inflammatory cytokines and chemokines in the lungs and brain. Three to five days following infection, K18-hACE2 mice begin to lose weight and become lethargic with laboured breathing All die within seven days. These observations support that transgene expression of hACE2 in epithelial cells can convert moderate SARS-CoV infection into a fatal disease.

Furthermore, a recent article published in Nature by Shi Zhengli's team showed that, like SARS-CoV, SARS-CoV-2 also enters human cells through ACE2. This supports the use of the K18-hACE2 transgenic mouse model for COVID-19 research.

Alternatively, K18-hACE2 mice can be obtained from the Jackson Laboratory, Bar Harbor, Me. 04609, USA.

80 mice are treated in 10 groups of 8 mice each as shown in the following table I

Vitamin D₃ (Vit. D3) is administered as intramuscular injection once per week shortly after birth.

Resveratrol (RSV) in form of an aqueous phospholipid formulation comprising 2 wt.-% of resveratrol and 13 wt.-% of phospholipids obtained from PlantaCorp GmbH, Hamburg, Germany, is dissolved in the food (AIN-93G diet, cp. P. G. Reeves, The Journal of Nutrition, Volume 127, Issue 5, May 1997, Pages 838S-841S) of the mice.

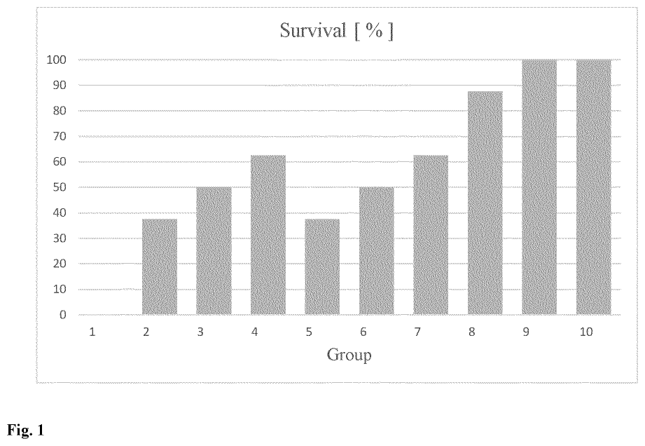

After 7 days the survival rate (n/8) of the groups 1 to 8 is monitored. The percentage of the survival rate of the different groups 1 to 10 is shown in the diagram of FIG. 1.

TABLE I

| | Treatment | | | Survival Rate | |
|---|---|---|---|---|---|
| Group | Active Ingredient | Amount | | n/8 | [%] |
| 1 | none | 0 | | 0 | 0 |
| 2 | Vit. D3 | 500 | IU | 3 | 37.5 |
| 3 | Vit. D3 | 750 | IU | 4 | 50 |
| 4 | Vit. D3 | 1000 | IU | 5 | 62.5 |
| 5 | RSV | 30 | mg | 3 | 37.5 |
| 6 | RSV | 45 | mg | 4 | 50 |
| 7 | RSV | 60 | mg | 5 | 62.5 |
| 8 | Vit. D3 + RSV | 500 IU | + 30 mg | 7 | 87.5 |
| 9 | Vit. D3 + RSV | 750 IU | + 45 mg | 8 | 100 |
| 10 | Vit. D3 + RSV | 1000 IU | + 60 mg | 8 | 100 |

### Example 2—Inhalation Formulation for p-MDI

A formulation according to the present invention can be made as follows:

1. Dispensing of Propellant, Ethanol and Water Charge Formulation Vessel with:

| | |
|---|---|
| Propellant HFA 134a | 97.5% by weight |
| ethanol | 2.4% by weight |
| water | 0.09% by weight |
| citric acid | 0.01% by weight |

2. Final Product Formulation

Add 24.0 g of resveratrol and 4.0 g vitamin D₃ to 50 g of the mixture of propellant-ethanol-water

Concentration of Formulation at this Process Step:

| | |
|---|---|
| Propellant | 60.93% |
| Ethanol | 1.5% |
| Water | 0.056% |
| Citric acid | 0.006% |
| Resveratrol | 30.0% |
| Vitamin D₃ | 5.0% |

Each actuation of the inhaler delivers 75 mg of solution and delivers 22.5 mg of resveratrol and 3,75 mg of vitamin D₃ from the mouthpiece.

### Example 3 Nasal Spray

52 mg of resveratrol, 525 mg of carboxymethyl beta-glucan (CM glucan) and 1.0 mg of vitamin D₃ (vit. D) are dissolved in a mixture of 80 ml of water and 20 ml of ethanol, which contains additionally aloe vera, zinc chloride, glycerol, fructose, and is buffered with dipotassium phosphate, sodium chloride and potassium phosphate.

The resulting solution is sprayed with a nasal spray bottle into a nostril while closing the opposite nostril to assure the dose. Each spray dose contains 14,71 mg of CM glucan, 1,45 mg resveratrol and 0,028 mg of vit. D.

### Example 4 Small Scale Trial

In a total of 10 patients, who do not feel sick, but who recently were tested positive for COVID-19, for example because they have entered from a high-risk country. They have not yet received any specific therapy, after appropriate information and consent, a new smear is taken, which is again positive. Thereafter, the patients are instructed to inhale resveratrol and vitamin D₃ in form of the formulation of Example 2 four times (5 puffs each) at intervals of 2 hours and inhale the active ingredients deeply into their lungs using a suitable p-MDI. After further 2 hours, another throat and nose swab is taken. The result of this virus test is negative. The smear is repeated the next day and is negative again.

### Example 5 Proof of Concept Study

The study shows the efficacy, safety, and tolerability of the combined, administration of 250 mg resveratrol and 2,000 I.U. vitamin D₃ twice daily and the topical (nose and throat spray) application of 6.0 mg resveratrol per day. About 100 adult asymptomatic patients, which are positive for SARS-CoV-2, are included in the study after a confirmatory test. The patients are randomly assigned in a double-blind study design to (i) oral administration of resveratrol and vitamin D₃ (40 patients), and (ii) resveratrol alone administered by nasal and throat spray and/or inhalation (40 patients) each for 2 weeks in both the verum arms, and (iii) placebo treatment in the control arm (20 patients) . Subsequently, the patients have a daily SARS-CoV-2 tests until day 7 and then at End of Treatment day 14. The study demonstrates that patients receiving the combination of oral resveratrol and vitamin D₃ or the resveratrol application by nasal and throat spray and/or inhalation show significantly earlier a negative SARS-CoV-2 test as compared to patients without treatment.

1. A composition consisting essentially of an effective amount of a source of a resveratrol derivative, an effective amount of a vitamin D, and an acceptable excipient for the prevention of an acute respiratory distress syndrome and/or an acute lung injury caused by the severe acute respiratory syndrome coronavirus 2 (SARS-CoV-2).

2. A composition according to claim 1, wherein the source of a resveratrol derivative is selected from the group consisting of synthetically produced hydroxylated stilbenes, liposomal resveratrol and extracts of plants comprising high amounts of resveratrol.

3. A composition according to claim 1 or 2, wherein the source of a resveratrol derivative is selected from the group consisting the resveratrol of formula (IA) and hydroxyresveratrol of formula (IB) 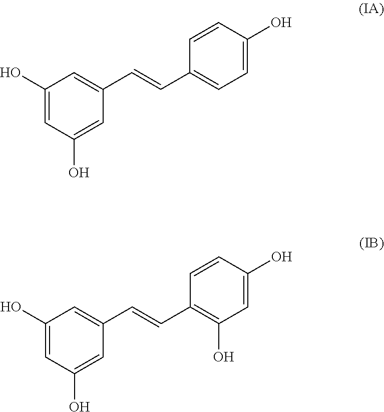 [SMILES: Oc1ccc(/C=C/c2cc(O)cc(O)c2)cc1 ; Oc1cc(O)cc(/C=C/c2ccc(O)cc2O)c1]
and an extract of polygonum cuspidatum or polygonum aviculare.

4. A composition according to one of claims 1 to 3, wherein the vitamin D is selected from the group consisting of vitamin D₃ and calcitriol.

5. A composition according to one of the claims 1 to 4, for the prevention of an acute respiratory distress syndrome and/or an acute lung injury caused by SARS-CoV-2 by inhibiting the NLRP3 inflammasome.

6. A composition according to one of claims 1 to 5, which comprises 0.01 to 10% by weight, preferably 0.05 to 5% by weight of a source of a resveratrol derivative with respect to the total weight of the composition.

7. A composition according to one of claims 1 to 6, which comprises 0.020 (800 IE) to 0.50 (20′000 IE) mg of a vitamin D.

8. A composition according to one of the claims 1 to 7, which is in a form suitable for oral administration.

9. A composition according to one of the claims 1 to 8, for the prevention of a still asymptomatic or mild symptomatic viral infection of the respiratory system, wherein pharmaceutically effective amounts of a source of a resveratrol derivative and of a vitamin D are administered by inhalation, preferably with a nasal, mouth, or throat spray.

10. A composition according to one of the claims 1 to 9, wherein one or more effective doses of a source of a resveratrol derivative and of a vitamin D are administered by inhalation sequentially each followed by a viral test until said test is negative.

11. A composition according to one of the claims 1 to 10, which is in a form suitable for inhalational administration by an inhaler selected from the group consisting of nasal sprays, oral sprays, propellant-free soft mist inhalers, in particular the Respimat®, dry powder inhalers (DPI), and metered dose inhalers (MDI).

12. A kit of parts for the preparation of a composition in accordance with the claims 1 to 11 essentially consisting of
(i) a first compartment containing a composition, which is in a form suitable for oral, local or inhalative administration, comprising an effective amount of a source of a resveratrol derivative, and an acceptable excipient;
(ii) a second compartment containing a composition, which is in a form suitable for oral, local, inhalative or parenteral administration, comprising an effective amount of a vitamin D, and an acceptable excipient;
(iii) optionally a leaflet describing the dosage and administration of each of the pharmaceutical compositions (i) and (ii).

13. A kit of parts according to claim 12 comprising
(i) a first compartment containing a composition, which is in a form suitable for oral, local or inhalative administration, comprising 0.001 to 10% by weight, preferably 0.05 to 5% by weight of a source of a resveratrol derivative, and an acceptable excipient;
(ii) a second compartment containing a pharmaceutical composition, which is in a form suitable for oral, local, inhalative or parenteral administration, comprising 0.020 mg (800 IE) to 0.5 mg (20′000 IE) of a vitamin D, and an acceptable excipient;

14. A method for the prevention of an acute respiratory distress syndrome and/or an acute lung injury caused by SARS-CoV-2 in a patient in need of such treatment or prevention, which method comprises administering to the patient in need thereof simultaneously or subsequently preventively effective amounts of
(a) an effective amount of a source of a resveratrol derivative, and
(b) an effective amount of a vitamin D.

15. Resveratrol for the prophylactic treatment of a still asymptomatic or mild viral infection of the respiratory system by the coronavirus 2 (SARS-CoV-2), wherein an effective amount of said resveratrol is administered locally to the to the respiratory system, preferably by inhalation, in particular by a nasal or throat spray.
